(19) United States
(12) Patent Application Publication
(10) Pub. No.: US 2024/0065677 A1
(43) Pub. Date: Feb. 29, 2024

(54) Body Fluid Collection Device

(71) Applicant: SHENZHEN HEALTH SWIFTECH CO., LTD., Shenzhen (CN)

(72) Inventors: Chih-min CHIU, Shenzhen (CN); Szu-han FANG, Shenzhen (CN); Jiaming ZHAO, Shenzhen (CN); Hsien-da HUANG, Shenzhen (CN)

(21) Appl. No.: 18/238,447

(22) Filed: Aug. 25, 2023

(30) Foreign Application Priority Data
Aug. 26, 2022 (CN) 2022110338678

(51) Int. Cl. A61B 10/00; A61B 5/15

(52) U.S. Cl. CPC A61B 10/0045; A61B 5/150343; A61B 10/0051; A61B 10/0058; A61B 10/0064; A61B 10/007; A61B 2010/0067; A61B 2010/0074; A61B 2010/0077

(57) ABSTRACT

The present disclosure relates to a body fluid collection device (1), the body fluid collection device (1) comprises a tube body (10), wherein the tube body (10) is used to contain the collected body fluid and is provided with an opened end (12); a tube plug (20), wherein the tube plug (20) is configured to be able to hermetically close the opened end (12) of the tube body (10); and a filter (30), wherein the filter (30) is arranged in the tube body (10) for filtering out specific components from a body fluid to be collected, wherein the filter (30) is configured to be capable of being attached to the tube plug (20) at the open end, and the tube plug (20) is hermetically connected to the filter (30).

### CROSS-REFERENCE TO RELATED APPLICATION

The present disclosure claims the priority to the Chinese patent application with the filing No. 2022110338678 filed with the Chinese Patent Office on Aug. 26, 2022, and entitled “Body Fluid Collection Device”, the contents of which are incorporated herein by reference in entirety.

### TECHNICAL FIELD

The present disclosure relates to a body fluid collection device.

### BACKGROUND ART

Various body fluid collection devices are known in practice for storing body fluid, such as blood, to be analyzed or tested. This kind of body fluid collection device can be a vacuum-type body fluid collection device, for example, a vacuum collection tube, such as a vacuum blood collection tube. However, such a body fluid collection device is usually a full body fluid collection device. Since only part of the components in the body fluid need to be analyzed or detected in some cases, the collected body fluid needs to be processed before detection or analysis can be performed, which will cost extra time and require specific processing equipment.

For example, along with the rapid development of high-throughput sequencing technology, detection items based on cell-free DNA in blood and pathogen detection items in blood are on the rise gradually, such as non-invasive prenatal free DNA screening, early cancer screening, pathogen nucleic acid detection, etc. The substances to be detected for these testing items respectively are cffDNA (cell-free fetal DNA contained in the peripheral blood of pregnant women), ctDNA (a cell-free DNA that is different from normal cell DNA and released by apoptosis of tumor cells contained in the peripheral blood of cancer patients), and pathogenic nucleic acid in the blood. The content of this cell-free DNA and nucleic acid to be detected in the blood is extremely low, so removing the interfering components will improve the detection sensitivity. As for common NIPT and cancer NGS experiments, the differential centrifugation is used to absorb supernatant plasma to remove white blood cells, but a small number of white blood cells cannot be seen via the naked eye, which may easily lead to unintentional maloperation that does not absorb the small number of white blood cells. As for common pathogenic nucleic acid detection, the step of using chemical reagents to remove the host is usually carried out before nucleic acid extraction, including the differential lysis method and methylation method, but such method takes a long time and is easy to cause sample loss or introduce contamination. The differential lysis method, the most commonly used method, is easy to “accidentally damage” tenericutes, and the operation is cumbersome and time-consuming. In addition, because only a small part of hospitals have NGS (Next-Generation Sequencing) qualification, in the case of transporting samples to a qualified third-party NGS laboratory, some white blood cells will still undergo apoptosis due to normal metabolism during blood transportation and be released as human nucleic acid becoming a source of interference.

### SUMMARY

Therefore, the object of the present disclosure is to provide a body fluid collection device, which can collect body fluid from which some components in the body fluid have been removed.

Body fluid can be understood herein as the liquid contained in the human body or animal body, which can be composed of water and inorganic and organic substances dissolved in water, etc. In this case, exemplarily, the body fluid can include blood, cerebrospinal fluid, gastric juice, digestive juice, semen, saliva, sputum, tears, sweat, urine, vaginal secretion, and the like. In addition, body fluid can also include pleural hydrops, peritoneal hydrops, pericardial hydrops, joint hydrops, cystic hydrops of various organs, etc., which are produced in a state of illness. The mixture of the above-mentioned liquid contained in the animal body and the external additional liquid (such as lavage fluid, sample retention fluid, etc.) used for its extraction, dilution, detection, or analysis, etc. can also be understood as body fluid.

According to the present disclosure, the object is achieved as follows. A body fluid collection device is proposed, which has: a tube body, wherein the tube body is configured to accommodate the collected body fluid and has an opened end; a tube plug, wherein the tube plug is configured to hermetically seal the opened end of the tube body; and a filter, the filter is arranged in the tube body for filtering specific components from the body fluid to be collected, wherein the filter is configured to be capable of being attached to the tube plug at the open end, and the tube plug is hermetically connected with the filter. For example, for blood, for the purpose of detection and analysis, inflammatory substances, excess sugar, fat, etc. can be filtered out from the collected blood in a targeted manner. For different body fluids, the desired specific components can be filtered out as needed.

According to an embodiment of the present disclosure, the tube plug has a first section, a second section, and a third section with decreasing diameters in sequence, wherein the outer diameter of the first section is larger than the outer diameter of the tube body, the second section is configured to form an interference fit with the tube body, and the third section is configured to form an interference fit with the filter. The tube plug can be made, for example, of medical rubber, such as butyl rubber.

According to an embodiment of the present disclosure, the tube body is provided with a step at one side of the opened end, wherein the step can be vertical or inclined relative to the longitudinal axis of the tube body.

According to an embodiment of the present disclosure, the end portion of the filter connected to the tube plug is provided with a flange, wherein the flange has a first sub-flange protruding radially outward from the cylinder body of the filter, and the first sub-flange is configured to abut against the step.

According to an embodiment of the present disclosure, the flange has a second sub-flange protruding radially inwardly from the cylinder body of the filter, wherein a groove is provided between the second section and the third section of the tube plug, and the second sub-flange can engage with the groove.

According to an embodiment of the present disclosure, the tube body has an outer tube and an inner tube connected together via a bridging portion, wherein the axial length of the inner tube is less than the axial length of the outer tube, and the open side of the inner tube and the open side of the outer tube are spaced apart.

According to an embodiment of the present disclosure, an interval is formed between the outer tube and the inner tube, especially a negative pressure interval, preferably a vacuum interval.

According to an embodiment of the present disclosure, the outer tube is made of PET (polyethylene terephthalate), the bridging portion and the inner tube are made of PET or PP (polypropylene); or the inner tube and the outer tube are made of PE (polyethylene), PS (polystyrene), PC (polycarbonate), PA (polyamide), and borosilicate glass.

According to an embodiment of the present disclosure, the inner wall of the tube body is siliconized (silicified), and a first material layer for body fluid is provided, and/or the inner wall of the filter is siliconized, and a second material layer for body fluid is provided.

According to an embodiment of the present disclosure, the filter membrane is provided at the second end of the filter opposite to the open end, wherein the filter membrane is separated from the bottom of the tube body by a predetermined distance.

According to an embodiment of the present disclosure, the body liquid collection device has a cover, wherein the cover covers the tube plug and is connected with the tube plug in a force transmission manner.

According to an embodiment of the present disclosure, the inner wall of the cover is provided with an annular protrusion, wherein the inner diameter of the cross-section of the annular protrusion gradually decreases from the open side of the cover towards the top of the cover, and the first section of the tube plug can be accommodated between the top of the cover and the annular protrusion.

According to an embodiment of the present disclosure, at least one of the two end sides of the tube plug in the axial direction is provided with a recess.

According to an embodiment of the present disclosure, a functional substance is arranged in the tube body, such as a functional fluid or a functional colloid, such as a preservation solution or colloidal gold.

According to an embodiment of the present disclosure, the body fluid to be collected is blood, cerebrospinal fluid, gastric juice, digestive juices, semen, saliva, sputum, tears, sweat, urine, vaginal secretion, hydrops, or a liquid mixture containing the above-mentioned body fluids, etc.

The beneficial effects of the present disclosure are at least as follows. Specific compositions can be filtered out by filter from the body fluid, thereby realizing better analysis or detection effect; the filter can be removed without affecting the use of collected body fluid samples; it has a protective effect during transportation and is not easy to break; and the tube plug is integrally formed, which is easy to produce, has good air tightness and is easy to be punctured. Especially when the cylinder body of the filter is provided with a sub-flange protruding radially outward, in the case of press-fitting, the sub-flange can abut against the step of the tube body, thereby providing sufficient supporting force to make the tube plug snap into the opening at the open side of the cylinder body of the filter.

### BRIEF DESCRIPTION OF DRAWINGS

The present disclosure is explained schematically below with reference to the drawings.

FIG. 1 schematically shows a body fluid collection device according to the present disclosure;

FIG. 2 schematically shows the tube body of the body fluid collection device according to the present disclosure;

FIG. 3 schematically shows a filter of a body fluid collection device according to the present disclosure;

FIG. 4 schematically shows a tube plug of a body fluid collection device according to the present disclosure;

FIG. 5 schematically shows a perspective view of the cover of the body fluid collection device according to the present disclosure;

FIG. 6 schematically shows a sectional view of a cover of a body fluid collection device according to the present disclosure; and

FIG. 7 schematically shows another embodiment of the tube body of the body fluid collection device according to the present disclosure.

### DETAILED DESCRIPTION OF EMBODIMENTS

Preferred embodiments of the present disclosure will be described in more detail below with reference to the drawings. Although preferred embodiments of the present disclosure are shown in the drawings, it should be understood that the present disclosure can be implemented in various forms and should not be limited to the embodiments set forth herein. Rather, these embodiments are provided so that the present disclosure will be more thorough and complete, and can fully convey the scope of the present disclosure to those of skill in the art.

In the following description, for the purposes of illustrating various embodiments of the present disclosure, certain specific details are set forth so as to provide a thorough understanding of the various embodiments of the present disclosure. However, people of skill in the art will recognize that the embodiments can be practiced in the cases without one or more of these specific details. In other instances, well-known devices, structures, and techniques associated with the present disclosure are not shown or described in detail so as to avoid unnecessarily obscuring the description of the embodiments.

Unless the context requires otherwise, throughout the specification and claims, the word “include/comprise” and variations thereof, such as “contain” and “have” shall be understood as an open and inclusive meaning, that is, it should be interpreted as “include, but not limited to”.

In addition, terms such as first and second used in the specification and claims are only used to distinguish each object for the clarity of description, and do not limit the size or other order of the objects described therein.

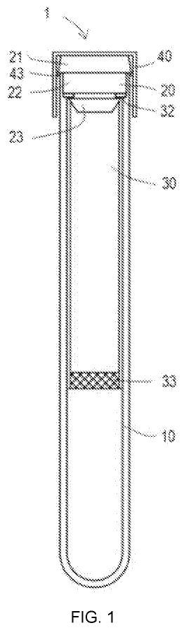

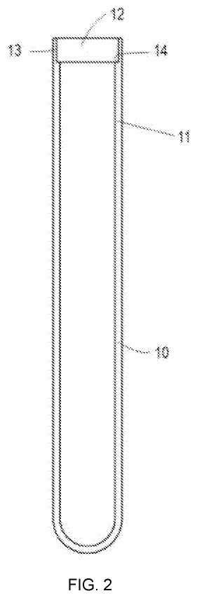

In FIG. 1, a body fluid collection device 1 according to an embodiment of the present disclosure is schematically shown, wherein the body fluid collection device 1 has a tube body 10, a tube plug 20, and a filter 30. The tube body 10 is configured to accommodate the collected body fluid, which is of a cylindrical structure with one end opened, and has an opened end 12 and a bottom end opposite to the opened end 12. The bottom end can be a closed bottom end. Preferably, the tube body 10 is made of an at least partially transparent material so that the volume of body fluid that has collected in the tube body 10 can be seen. Further, preferably, a scale can be provided on the outer peripheral side of the tube body 10, so that the volume of body fluid collected in the tube body 10 can be known specifically. Furthermore, it is also shown in FIG. 2 that the tube body 10 can be provided with a step 14 in the section of its open side, which will be further explained below.

The tubular body 10 can be an integrally formed part, such as a molded part. For example, the tube body 10 can be produced by injection molding of PET material.

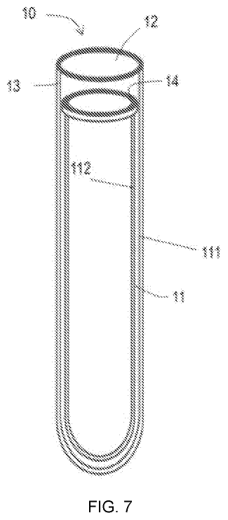

Alternatively, referring to FIG. 7, the main body 11 of the tube body 10 can include an outer tube 111 and an inner tube 112, wherein the outer tube 111 and the inner tube 112 are connected together by a bridging portion. Preferably, the inner tube 112 and the bridging portion are integrally formed. The inner tube 112 has an axial length smaller than that of the outer tube 111, and the open side of the inner tube 112 is spaced apart from the open side of the outer tube 111, thereby forming the above-mentioned step 14. The section 13 above the step can be configured to engage with the tube plug 20. The inner wall of the outer tube 111 and the outer wall of the inner tube 112 can be spaced apart from each other. Preferably, a negative pressure interval or vacuum interval is formed between the outer tube 111 and the inner tube 112. With this kind of structure, the negative effects of external factors (such as heat, gas, etc.) on the collected body fluid can be better prevented, thereby keeping the quality of the collected body fluid longer and at the same time extending the validity period of the body fluid collection device. In addition, this double-layer structure also has a certain protective effect during transportation, thereby being not easy to break. Here, for example, the outer tube 111 and the inner tube 112 are molded separately, and then assembled together in a vacuum environment, such that an isolation layer with a U-shaped section is formed between the inner wall of the outer tube 111 and the outer wall of the inner tube 112. For example, the outer tube 111 can be made of PET, and the bridging portion and inner tube can be made of PP or PET. Certainly, the outer tube 111 and the inner tube 112 can also be made of any other suitable material, for example, PE (polyethylene), PS (polystyrene), PC (polycarbonate), PA (polyamide) and borosilicate glass.

When it is necessary, the inner wall of the tube body 10 that is in direct contact with the body fluid can be siliconized to prevent liquid from hanging on the wall. After siliconizing the inner wall of the tube body 10 that is in direct contact with the body fluid, different functional layers, i.e., additional material layers, can be provided on the siliconized inner wall as required. For example, when the body fluid collected is blood, the functional layer can be a coagulant or an anticoagulant, wherein the coagulant can accelerate blood coagulation and shorten the test time; and the anticoagulant can prevent blood coagulation, and the anticoagulant can be, for example, heparin, ethylenediaminetetraacetic acid (EDTA), sodium oxalate, sodium citrate, etc.

In order to seal the opened end 12 of the tube body 10, the body fluid collection device 1 is also provided with a tube plug 20 which hermetically closes the tube body 10, so as to maintain a negative pressure condition within the tube body 10. To this end, the tube plug 20 can be in interference fit with the tube body 10. For example, in one embodiment, the outer diameter of one section of the tube plug 20 is slightly larger than the inner diameter of the tube body 10, and the tube plug 20 is configured to be elastically deformable.

The tube plug 20 is penetrable for a body fluid collection needle. For example, the plug 20 can be made of rubber, e.g. medical rubber, such as butyl rubber. In order to make it easier for the collection needle to puncture the tube plug 20, the tube plug 20 can be provided with a central recess 25, 26 on at least one of the two end sides thereof in the axial direction, as can be seen in FIG. 4.

The body fluid collection device 1 is also provided with a filter 30 in the tube body 10 for filtering out certain components from the body fluid to be collected. The filter 30 can also be in a cylindrical structure, one end of the cylinder body 31 thereof is an open end, and the opposite end is provided with a filter membrane 33, wherein the filter membrane 33 is configured to prevent unwanted specific components in the collected body fluid from entering the space of the tube body 10 below the filter membrane 33. For this reason, when the body fluid collection device 1 is assembled, the filter membrane 33 is spaced apart from the bottom of the tube body 10, and the spaced distance is designed so as to enable the accommodation of at least the volume of body fluid required to complete the test. The filter membrane 33 can be a separate member relative to the cylinder body 31 of the filter 30, which is attached to the end side of the cylinder body 31 through subsequent processing, for example, fixedly connected with the end side of the cylinder body 31 through an ultrasonic heat bonding process.

The filter membrane can have a substrate selected from those provided with Leukosorb membranes, cellulose triacetate, cellulose acetate, cellulose glass, quartz cellulose, cellulose nitrate, regenerated cellulose, or nylon. Certainly, other filter membranes are also feasible.

The axial length of the cylinder body 31 is designed so that after the collection needle has penetrated the tube plug 20 and entered the filter 30, the top of the collection needle cannot reach the filter membrane 33 of the filter 30, so as to prevent the collection needle from inadvertently puncturing the filter membrane 33.

The inner wall of the cylinder body 31 of the filter 30 can likewise be siliconized and provided with a functional layer as desired. For example, when the collected body fluid is blood, the functional layer can be a procoagulant or an anticoagulant, wherein the procoagulant accelerates the coagulation of the blood and shortens the time of the test, and the anticoagulant prevents the blood from coagulating.

As can be seen from FIG. 1, the end portion of the tube plug 20 protrudes into a section of the open end side of the filter 30, so that the opened end of the filter 30 is also sealed by the tube plug 20. Therefore, the tube plug 20 can be used to seal the tube body 10 and the filter 30 at the same time, which is advantageous for reducing the number of parts constituting the body fluid collection device 1. For this purpose, referring to FIG. 4, the tube plug 20 can have a first section 21, a second section 22 and a third section 23 which are coaxially arranged with decreasing diameters in sequence, wherein the outer diameter of first section 21 is larger than that of the tube body 10, the second section 22 is configured to form an interference fit with the tube body 10, and the third section 23 is configured to form an interference fit with the filter 30. A sealing connection is formed between the second section 22 and the tube body 10 and between the third section 23 and the filter 30 through the above interference fits.

Preferably, as can be seen from the FIG. 4, the first section 21 and/or the third section 23 can be constructed in a truncated conical shape, wherein a small top surface of the first section 21 faces away from the third section 23 and/or a small top surface of the third section 23 faces away from the first section 21, which facilitates mounting. Certainly, rounding or chamfering can be performed only at the edges of the opposite distal end surfaces of the first section 21 and/or the third section 23, that is, the above-mentioned truncated conical shape only extends over the partial axial length of the first section 21 and/or third section 23, and the remaining axial sections can be cylindrical.

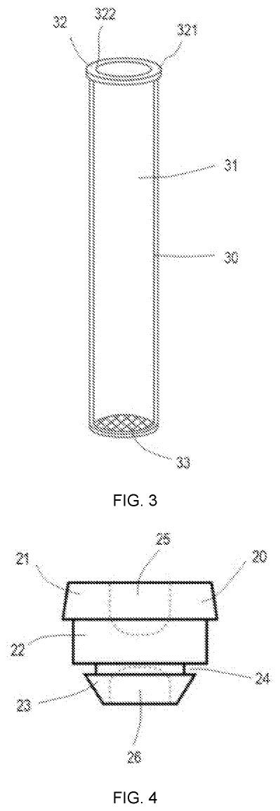

In order to further ensure that when the tube plug 20 is pulled out, the tube plug 20 can be taken out from the tube body 10 together with the filter 30, the tube plug 20 and the filter 30 can be constructed in the following manner. That is, a groove 24 is provided between the second section 22 and the third section 23 of the tube plug 20, and the end portion of the cylinder body of the filter 30 connected to the tube plug 20 is provided with a flange 32 (see FIG. 3), wherein the flange 32 has a sub-flange 322 that protrudes inwardly in a radial direction from the cylinder body 31 of the filter 30, and the sub-flange 322 can engage into the groove 24. Thus, the inner diameter of the sub-flange 322 is less than the outer diameter of the portion of the third section 23 of the tube plug 20 close to the groove 24. Through the interference fit between the third section 23 of the tube plug 20 and the cylinder body 31 of the filter 30 and the snap-fit of the groove 24 of the tube plug 20 and the sub-flange 322 of the cylinder body 31 of the filter 30, the undesired separation of the tube plug 20 from the filter 30 when pulling out the tube plug 20 is reliably prevented.

Additionally, the flange 32 can be provided with a sub-flange 321 protruding radially outward from the cylinder body 31 of the filter 30, wherein the sub-flange 321 is configured to abut against the step 14, preferably hermetically, which is advantageous for installation. At this time, the end surface of the second section 22 of the tube plug 20 can hermetically abut against the sub-flange 321 of the filter 30. The outer diameter of the sub-flange 321 is not larger than the outer diameter of the step 14. Through this solution, the filter can be clamped between the tube plug and the tube body under the condition of ensuring the negative pressure inside the body fluid collection device 1, thereby preventing the filter from wobbling.

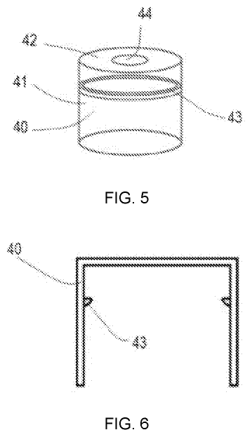

The body fluid collection device 1 can also have a cover 40, wherein the cover 40 covers the tube plug 20. The cover can be used to protect the tube plug and prevent body fluid from splashing when the tube plug is pulled out. The cover 40 can be of different colors for distinguishing different types or uses of body fluid collection devices. As shown in FIG. 5, the top 42 of the cover 40 is provided with an opening 44 to facilitate the body fluid collection needle inserting into and piercing through the tube plug to enter the tube. In order to prevent dirt from contaminating the part of the rubber plug that is not covered by the top 42 of the cover, in an embodiment not shown, the top 42 of the cover 40 can also be provided with a sealing membrane covering the opening 44, wherein the sealing membrane is removably affixed at the top 42 of the cover 40.

The cover 40 can be connected to the tube plug 20 in a force transmission manner, so that the force acting on the cover 40, in particular the axial force, can be transmitted to the tube plug 20. In other words, the cover 40 and the tube plug 20 are arranged such that at least the axial force can be transmitted. In the example shown, referring to FIG. 5 and FIG. 6, the inner wall of the peripheral portion 41 of the cover 40 is provided with an annular protrusion 43, wherein the inner diameter of the cross-section of the annular protrusion 43 gradually decreases in a direction from the open side of the cover 40 toward the bottom 42 of the cover 40, wherein the first section 21 of the tube plug 20 that can protrude beyond the outer wall of the tube body 10 is between the bottom 42 of the cover 40 and the annular protrusion 43, so that the annular protrusion 43 is below the first section 21 of the tube plug 20, so that when the cover 40 is pulled out, the cover 40 is removed from the tube body 10 simultaneously together with the tube plug 20 and the filter through the interaction between the annular protrusion 43 of the cover 40 and the first section 21 of the tube plug 20.

Certainly, the cover 40 can also be in a force transmission connection with the tube plug 20 in other ways, such as threaded connection, adhesive bonding and the like.

Functional fluid can also be arranged in the tube body 10 of the body fluid collection device 1. For example, when the body fluid is blood, the functional fluid can be a DNA preservation solution to maintain the quality of the filtered body fluid temporarily stored in the collection device.

When installing the above-mentioned components of the body fluid collection device 1, in a vacuum environment, when the cylindrical body of the filter is provided with a flange protruding radially outwardly, the filter is first put into the tube body, and then the tube plug is pressed in so that the second section and third section of the tube plug occupy the correct positions relative to the tube body and the cylinder body of the filter, respectively, and the radially inwardly protruding flange of the cylinder body enters the groove of the tube plug. Then the cover is covered on the tube plug. When the cylinder body of the filter is only provided with a radially inwardly protruding flange, in a vacuum environment, the tube plug and the cylinder body of the filter can be assembled first so that the radially inwardly producing flange of the cylinder body enters the groove of the tube plug. The tube plug is then inserted into the tube body together with the cylinder body, and the cover is then placed on the tube plug. It should be noted that the above installation steps are only exemplary, and can also be adjusted according to needs, that is, the installation is completed in a sequence different from the steps mentioned above, as long as the installation can be completed to realize the predetermined function.

An exemplary use of the body fluid collection device 1 according to the present disclosure is described below. The body fluid collection device 1 can be used to collect blood for NGS testing. During blood collection, it is necessary to filter out white blood cells in the blood while retaining pathogenic microorganisms in the blood. In this kind of body fluid collection device 1, the inner wall of the cylinder body of the filter is siliconized and provided with an evenly coated anticoagulant, and the inner wall of the tube body is siliconized and provided with an evenly coated anticoagulant. Free DNA preservation solution is reserved at the bottom of the tube body. The filter membrane can intercept the white blood cells in the blood in the filter, and allow other components in the blood to pass through, so that there is no need to use separation gel, thereby avoiding contamination of the collected blood by precipitates. The blood collected by the body fluid collection device 1 of the present disclosure can effectively remove the interfering nucleic acid caused by the apoptosis of white blood cells because the white blood cells in the blood have been filtered out during the collection process. For NIPT and tumor NGS, filtering out white blood cells during blood collection can avoid absorbing of white blood cells when absorbing plasma during differential centrifugation; and for nucleic acid detection experiments, white blood cells are removed from the blood during blood collection, which omits the experiment step of removing host and improves the accuracy of the results.

It should be noted that the features or feature combinations of the above-described device according to the present disclosure and the features and feature combinations mentioned in the drawings and/or only shown in the drawings can not only be used according to the correspondingly given combination, other combinations or single use can also be adopted without departing from the scope of the present disclosure.

The present disclosure has been described through the above-mentioned embodiments, but it should be understood that the above-mentioned embodiments are only for the purpose of illustration and description, and are not intended to limit the present disclosure to the scope of the described embodiments. Those of skill in the art should understand that more variations and modifications can be made according to the teachings of the present disclosure, and these variations and modifications all fall within the protection scope of the present disclosure.

What is claimed is:

1. A body fluid collection device, wherein the body fluid collection device comprises:
a tube body, wherein the tube body is configured to accommodate a collected body fluid and is provided with one opened end;
a tube plug, wherein the tube plug is configured to be able to hermetically close the opened end of the tube body; and
a filter, wherein the filter is arranged in the tube body and configured to filter out specific components from a body fluid to be collected,
wherein
the filter is configured to be capable of being attached to the tube plug at an open end, and the tube plug is hermetically connected to the filter.

2. The body fluid collecting device according to claim 1, wherein the tube plug has a first section, a second section, and a third section with decreasing diameters in sequence, wherein an outer diameter of the first section is larger than an outer diameter of the tube body, the second section is configured to be able to form an interference fit with the tube body, and the third section is configured to be able to form an interference fit with the filter.

3. The body fluid collection device according to claim 2, wherein the tube body is provided with a step on one side of the opened end.

4. The body fluid collection device according to claim 3, wherein an open end of the filter connected to the tube plug is provided with a flange, wherein the flange is provided with a first sub-flange protruding radially outwardly from a cylinder body of the filter, wherein the first sub-flange is configured to abut against the step.

5. The body fluid collection device according to claim 4, wherein the flange has a second sub-flange protruding radially inwardly from the cylinder body of the filter, wherein a groove is provided between the second section and the third section of the tube plug, and the second sub-flange can engage into the groove.

6. The body fluid collection device according to claim 3, wherein the tube body is provided with an outer tube and an inner tube connected together by a bridging portion, wherein an axial length of the inner tube is smaller than an axial length of the outer tube, and an open side of the inner tube is spaced apart from an open side of the outer tube.

7. The body fluid collection device according to claim 6, wherein a negative pressure interval is formed between the outer tube and the inner tube.

8. The body fluid collection device according to claim 6, wherein the outer tube is made of PET, and the bridging portion and the inner tube are made of PET or PP; or the outer tube and the inner tube are made of PE, PS, PC, PA, or borosilicate glass.

9. The body fluid collection device according to claim 1, wherein
an inner wall of the tube body is siliconized and is provided with a first material layer for body fluids, and/or
an inner wall of the filter is siliconized and is provided with a second material layer for body fluids.

10. The body fluid collection device according to claim 1, wherein a filter membrane is provided at a second end of the filter opposite to the open end, wherein the filter membrane is spaced at a predetermined distance from a bottom of the tube body.

11. The body fluid collection device according to claim 4, wherein the body fluid collection device is provided with a cover, wherein the cover covers the tube plug and is connected to the tube plug in a force transmission manner.

12. The body fluid collection device according to claim 9, wherein an inner wall of the cover is provided with an annular projection, wherein an inner diameter of a cross-section of the annular projection gradually decreases in a direction from an open side of the cover towards a top of the cover, wherein the first section of the tube plug is located between the top of the cover and the annular projection.

13. The body fluid collection device according to claim 1, wherein at least one of two end sides of the tube plug in an axial direction is provided with a recess.

14. The body fluid collection device according to claim 1, wherein a functional substance is arranged in the tube body.

15. The body fluid collection device according to claim 1, wherein the body fluid to be collected is blood, cerebrospinal fluid, gastric juice, digestive fluid, semen, saliva, sputum, tears, sweat, urine, vaginal secretion, hydrops, or liquid mixtures containing above-mentioned body fluids.

16. The body fluid collection device according to claim 2, wherein
an inner wall of the tube body is siliconized and is provided with a first material layer for body fluids, and/or
an inner wall of the filter is siliconized and is provided with a second material layer for body fluids.

17. The body fluid collection device according to claim 2, wherein a filter membrane is provided at a second end of the filter opposite to the open end, wherein the filter membrane is spaced at a predetermined distance from a bottom of the tube body.

18. The body fluid collection device according to claim 5, wherein the body fluid collection device is provided with a cover, wherein the cover covers the tube plug and is connected to the tube plug in a force transmission manner.

19. The body fluid collection device according to claim 2, wherein at least one of two end sides of the tube plug in an axial direction is provided with a recess.

20. The body fluid collection device according to claim 2, wherein a functional substance is arranged in the tube body.
